(19) United States
(12) Patent Application Publication
(10) Pub. No.: US 2006/0047259 A1
(43) Pub. Date: Mar. 2, 2006

(54) Fasteners with printed designs

(76) Inventors: Carol Erdman, West Chester, PA (US); Dane Jackson, Gilbertsville, PA (US)

Correspondence Address: Mark S. Leonardo, Esq.;Brown Rudnick Berlack Israels LLP, One Financial Center, Boston, MA, 02111, US

(21) Appl. No.: 10/930,603

(22) Filed: Aug. 30, 2004

(51) Int. Cl. A61F 13/15

(52) U.S. Cl. 604/389; 604/391

(57) ABSTRACT

A fastener having an extensible and non-extensible portion and dynamic indicia, wherein the indicia change shape or configuration upon extension of the fastener. The fastening tab includes an extensible layer adjacent a non-woven layer. The indicia may be placed intermediate or adjacent a plurality of non-woven layers and may be printed upon a non-woven or extensible layer.

### BACKGROUND

1. Technical Field

The present disclosure generally relates to disposable absorbent articles, and more particularly, to disposable absorbent articles having fasteners that include dynamic indicia.

2. Background of the Related Art

Absorbent articles such as, for example, disposable diapers, adult incontinent pads, sanitary napkins, pantiliners, incontinent garments, etc. are generally worn, in cooperation with garments and disposed against a body surface, etc., by infants or adult incontinent individuals. The absorbent article is employed to collect, absorb, etc. body fluid discharge, such as, for example, blood, menses, urine, aqueous body fluids, mucus, cellular debris, etc. For example, the absorbent article may be disposed between the legs of an individual adjacent a crotch area. The absorbent article is positioned with a garment and drawn into engagement with a body surface of the crotch area to collect fluid discharge.

As is known, absorbent articles typically include a fluid permeable coverstock for engaging the body surface, a fluid impermeable back sheet and an absorbent core supported therebetween. The backsheet serves as a moisture barrier to prevent fluid leakage to the garment. The absorbent core usually includes a liquid retention material that faces the body surface. The absorbent core can include loosely formed cellulosic fibers, such as wood pulp, for acquiring and storing fluid discharge.

Fastening of the absorbent articles with an individual requires the use of closure systems employing fastening tabs or fasteners. For example, in typical diaper-type garments, the garment is affixed to a wearer by attaching one or more fasteners that extend across the wearer's hips to hold the back and front halves of the garment to one another.

Some of these articles, however, suffer from performance drawbacks including poor fit with the body surface. These drawbacks can disadvantageously result in leakage and discomfort to the wearer. Attempts to overcome these drawbacks include providing fasteners having mechanical closure systems that employ an elastic portion for customizing the fit of an absorbent article to a particular individual. However, these fasteners can be stretched too tightly around the body of an individual. This can disadvantageously result in an improper fit, leakage and discomfort to the wearer.

It would therefore be desirable to overcome the disadvantages and drawbacks of the prior art by providing an absorbent article including a fastener that indicates fit of the absorbent article to an individual. It would be desirable if the absorbent article and its constituent parts are easily and efficiently manufactured. It would further be desirable if the design of absorbent article is aesthetically pleasing, especially to a child-wearer.

### SUMMARY

Accordingly, a fastener and manufacturing process are disclosed that includes a fastener tab or fastener having dynamic indicia. The fastener and its constituent parts are easily and efficiently manufactured.

Other objects and advantages of the present disclosure are set forth in part herein and in part will be obvious therefrom, or may be learned by practice of the present disclosure that is realized and attained by the instrumentalities and combinations pointed out in the appended claims for the devices and methods of the present disclosure consisting of its constituent parts, constructions, arrangements, combinations, steps and improvements herein shown and described.

In one particular embodiment, a diaper that includes a fastener tab is provided, in accordance with the principles of the present disclosure. A first crosswise portion of the fastening tab is extensible and includes a plurality of layers. There are indicia disposed with the first crosswise portion and adapted to change configuration upon extension of the fastening tab. During extension of the fastening tab, the indicia changes from a first configuration that indicates a desired fit to a second configuration that indicates a non-desired fit. A second crosswise portion of the fastening tab is non-extensible, and does not contain the indicia. The fastening tab includes an extensible layer adjacent at least one non-woven layer. The indicia may be placed intermediate or adjacent the non-woven layer. The indicia may be printed upon the non-woven or upon the extensible layer.

In another embodiment, a fastener adapted for use with an absorbent article is provided. The fastener includes a first crosswise portion that is extensible and includes a non-woven layer and an extensible layer. An indicia layer is disposed with or between the non-woven layer and the extensible layer, and is adapted to change configuration upon extension of the fastener. During extension of the fastening tab, the indicia changes from a first configuration that indicates a desired fit to a second configuration that indicates a non-desired fit, or changes from one pattern to another.

In another embodiment, an absorbent article including a pair of fasteners is provided. At least one of the fasteners includes indicia adapted to change configuration upon extension of the fastener so to indicate fit of the diaper to an individual. During extension of the fastener, the indicia changes from a first design that indicates a desired fit to a second design that indicates a non-desired fit. The fastener includes an extensible layer intermediate non-woven layers.

In another embodiment, a fit-indicating diaper including a pair of fasteners is provided. At least one of the fasteners includes dynamic indicia indicating a desired fit and a non-desired fit of the diaper to an individual. Each of the fasteners includes at least one non-woven substrate layer and at least one extensible substrate layer. The dynamic indicia are printed upon the non-woven substrate layer, or on the extensible substrate layer.

In another embodiment, a method of manufacturing the fastening tab is provided. The method includes the steps of providing a fastening tab having at least one non-woven substrate layer and at least one extensible substrate layer; and printing indicia on at least one of the non-woven substrate layer and the extensible substrate layer, wherein the indicia is aligned along a crosswise stretchable zone of the fastening tab. The indicia are adapted to change configuration upon extension of the fastening tab so to indicate fit of an absorbent article to an individual. During extension of the fastening tab, the indicia changes from a first configuration that indicates a desired fit to a second configuration that indicates a non-desired fit. The indicia may change shape so to indicate a desired or non-desired fit of an absorbent article to an individual.

### BRIEF DESCRIPTION OF THE DRAWINGS

The objects and features of the present disclosure are set forth with particularity in the appended claims. The present disclosure, as to its organization and manner of operation, together with further objectives and advantages may be understood by reference to the following description, taken in connection with the accompanying drawings, in which:

FIG. 1 is a plan view of an absorbent article including fasteners in accordance with the principles of the present disclosure;

FIG. 2A is an enlarged cutaway side view of the fastener shown in FIG. 1;

FIG. 2B is another enlarged cutaway side view of the fastener shown in FIG. 1;

FIG. 2C is another enlarged cutaway side view of the fastener shown in FIG. 1;

FIG. 3A is a top view of an unstretched position of the fastener shown in FIG. 1;

FIG. 3B is a top view of a stretched out position of the shown in FIG. 1; and

FIG. 4 is a perspective view of a manufacturing process for making the fastener shown in FIG. 1.

### DETAILED DESCRIPTION OF EXEMPLARY EMBODIMENTS

The exemplary embodiments of the fastener and methods of manufacture disclosed are discussed in terms of an absorbent article including a fastener tab or fastener that includes indicia of various parameters. The presently disclosed fastener includes dynamic indicia. It is contemplated that the fastener may be employed with absorbent articles such as disposable diapers, adult incontinent pads, feminine pads, sanitary napkins, incontinent garments, or other articles intended to avoid leakage, overflow, etc., of fluid discharge, such as, for example, blood, menses, urine, aqueous body fluids, mucus, cellular debris, etc.

In the discussion that follows, the term “body facing surface” refers to a portion of a structure that is oriented towards a body surface, and the “garment facing surface” refers to a portion of the structure that is oriented towards a garment and is typically opposing the body facing surface and may be referred to as such. As used herein, the term “body surface” refers to a portion of an individual's body that the absorbent article is disposed with for collecting, absorbing, etc. fluid discharge from the individual.

The following discussion includes a description of an absorbent article including a fastener, followed by a description of a method of manufacture in accordance with the present disclosure. Reference will now be made in detail to the exemplary embodiments of the disclosure, which are illustrated in the accompanying figures.

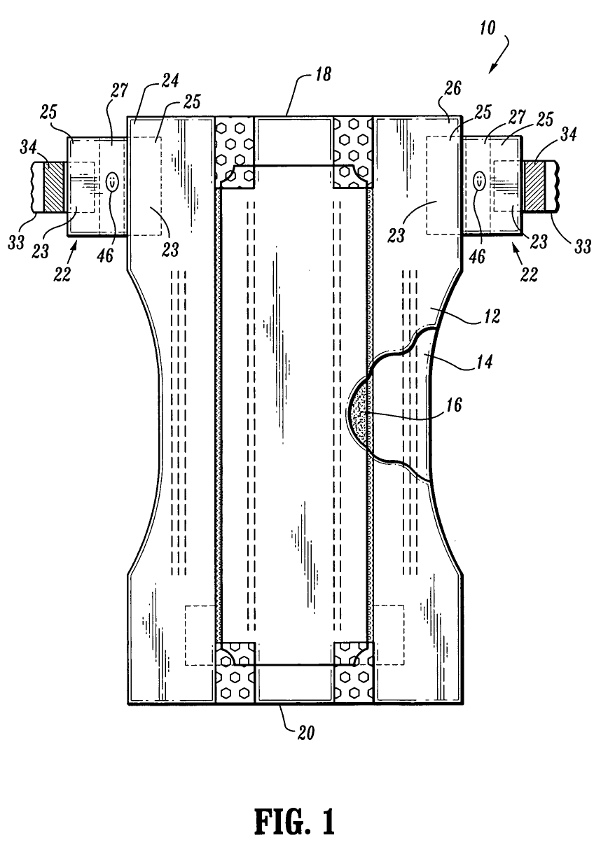

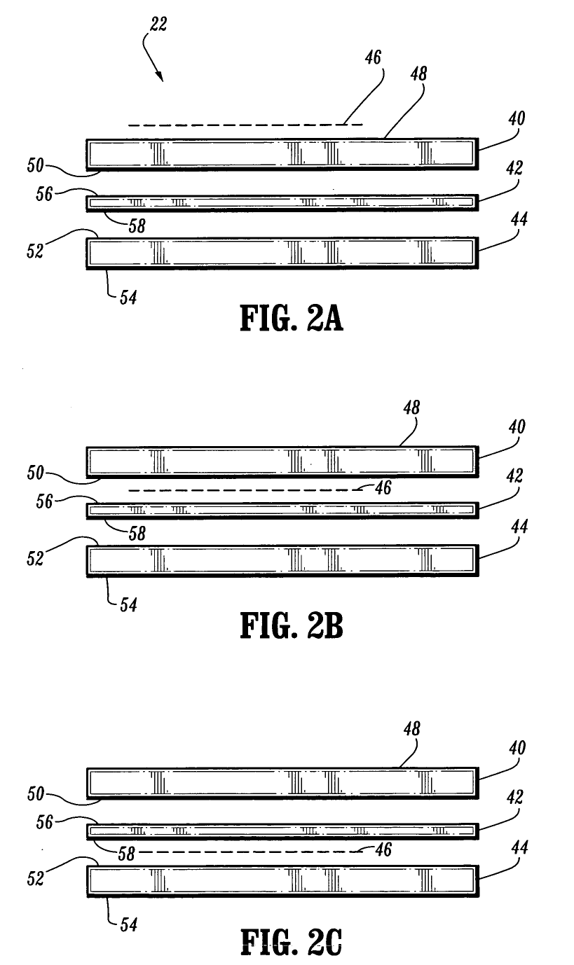

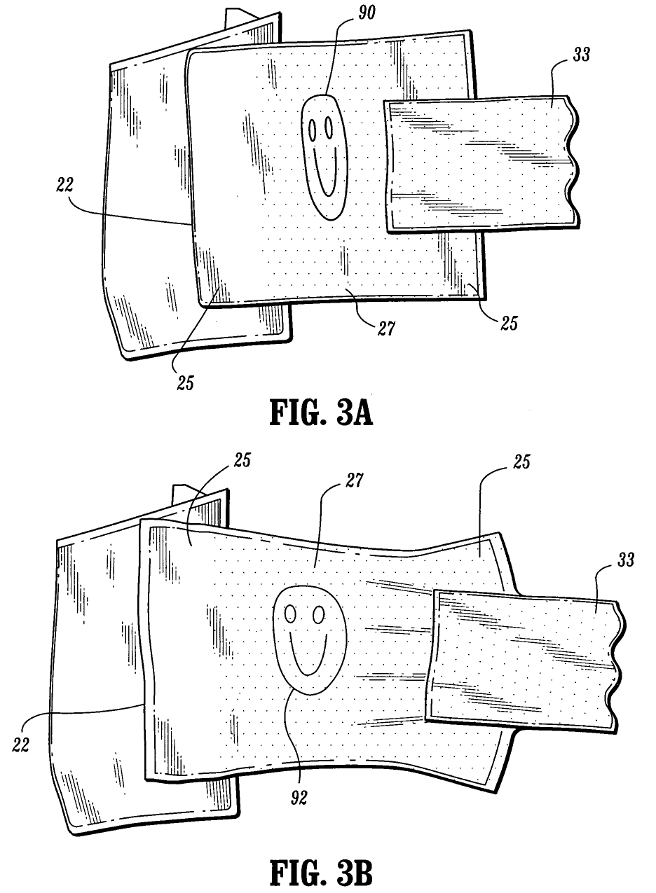

Turning now to the figures, wherein like components are designated by like reference numerals throughout the several views. Referring to FIGS. 1-4, there is illustrated a fastener tab(s) or fastener(s) 22, constructed in accordance with the principles of the present disclosure. Fastener 22 may be used to fasten absorbent articles, such as, for example diaper 10.

Diaper 10 includes a backsheet 12 bonded to a topsheet 14 and an absorbent core 16 disposed therebetween. Diaper 10 extends from a rear end 18, configured for engagement with a rear portion of a subject, to a front end 20, configured for engagement with a front portion of a subject, along a longitudinal axis x defined by diaper 10. Each of two fasteners 22 is made from partially elastic material and is adhesively bonded 23 to respective opposite sides 24, 26 of rear end 18. Fastener 22 is extensible in a direction along a transverse axis y relative to longitudinal axis x. Each of closure tabs 33 is attached distally to respective fasteners 22 by adhesive bonding 23. Closure tab 33 may comprise any hook-and-loop type material(s), adhesive, or other type of mechanical closure material 34 that is capable of holding diaper 10 on a wearer. Closure tab 33 operates by engaging with or adhering to a corresponding surface or object (not shown) located on the opposite end of the diaper 10, preferably front end 20.

Fastener 22 has a “zoned” stretch property, in that, only certain crosswise/lengthwise portions or zones 27 thereof will extend or stretch during use. Stretchable zone 27 of fastener 22 may comprise an elastic composite or laminate having an extensible layer 42, and at least one nonwoven layer 40 adjacent thereto. In the illustrative embodiments, stretchable zone 27 has extensible layer 42 intermediate or sandwiched between a first non-woven layer 40 and a second non-woven layer 44. Fastener 22 also includes non-extensible crosswise portions 25 that include layers 40, 42, 44. Portions 25 also include a layer of non-stretch or non-elastic film (not shown).

Extensible layer 42 may be, for example, a urethane based elastic or stretch film, such as EXTRAFLEX™ available from Tredegar Film Products of Richmond, Va. However, elastic layer 42 may also be another type of elastic film, a multidirectional elastic aggregate such as elastic webbing, netting, or scrim elastic, foam, strands or bands of suitable elastic materials, such as natural or synthetic rubber, spandex, LYCRA and elastic polymers. Other suitable elastics will be apparent to those skilled in the art in light of the present disclosure.

Non-woven layers 40, 44 may be, for example, spunbonded polypropylene, polyethylene, or other nonwoven material that encases elastic layer 42, protects the elastic layer 42, protects the wearer from uncomfortable exposure to the elastic layer 42 or provides other benefits. Other uses for the nonwoven layers 40, 44 will be apparent to those skilled in the art based on the present disclosure.

The bonding between layers 40, 42, 44 of fastener 22 may be accomplished using joining methods known in the art, such as, for example, compression bonds, heat bonds, ultrasonic bonds, adhesives and the like, or combinations of different bonding methods. The construction of such elastic laminates is known in the art, and a skilled artisan will be able to provide a suitable elastic laminate or other elastic design for fastener 22 without undue experimentation.

The present disclosure provides an improved fastener 22 that indicates various parameters, such as, for example, desired or non-desired fit of diaper 10 to a wearer. Examples of a desired fit include, for example, a properly sized fit, comfortable fit, or the like. Examples of a non-desired fit include, for example, an improperly sized fit, uncomfortable fit, a fit that is either too tight nor too loose, or the like. Fastener 22 includes an indicia layer 46. Indicia layer 46 changes as fastener 22 is stretched or extended in a crosswise or lateral direction. It is contemplated that the shape, color, size, or configuration of indicia 46 may change upon extension of fastener 22. It is further contemplated that indicia 46 may include printed indicia, or indicia applied by other techniques known in the art such as laser application, embossing, lithography, gravure, silk screen, or the like. Alternatively, indicia 46 may include dynamic indicia, that is, indicia that changes (shape, color, size, configuration, etc.) continuously. For example, dynamic indicia 46 may increase continuously (in size) in correspondence with increasing degrees of fastener extension so to indicate varying levels of desired and/or non-desired fit. Other advantages provided by the present disclosure include a diaper that is more aesthetically pleasing or more pleasing to the child-wearer since the indicia design is dynamic.

One example of how fastener 22 having indicia 46 indicates the desired and non-desired fit of diaper 10 to a wearer will now be described. In the case of a baby lying on his or her back, a caregiver usually places diaper 10 between the baby's legs, pulls front end 20 up between the legs and then attaches tabs 33 to rear end 18 of diaper 10, thereby forming a pant-like structure. In the case where diaper 10 is the correct size and thus fits the wearer in a desired manner, fastener 22 stretches only minimally around the wearer's body and thus indicia 46 printed on fastener 22 does not change substantially. For example, fastener 22 that is not stretched substantially has printed thereon indicia 46 that include a first design such as smiley face 90 that indicates a desired, i.e., properly sized, fit of diaper 10 to the wearer. In the case where diaper 10 is, for example, too small and thus does not fit the wearer in a desired manner, fastener 22 stretches substantially (too tightly) around the wearer's body and thus indicia 46 printed on fastener 22 changes configuration. For example, as fastener 22 stretches, smiley face 90 changes to a second design different than the first design, or stretched-out smiley face 92, which indicates an non-desired fit of diaper 10 to the wearer. In this way, fastener 22 with fit-indicating indicia 46 advantageously alerts a caregiver that it is time to go up to the next size of diaper 10.

Indicia 46 are preferably printed on fastener 22 along stretchable zone 27 thereof. Indicia 46 may be placed on, upon, intermediate, between, or adjacent nonwoven layers 40, 44 or extensible layer 42. For example, indicia 46 may be printed on an outer surface(s) 48, 54 or an inner surface(s) 50, 52 of first or second non-woven layers 40, 44. Alternatively, indicia 46 may be printed on either of side(s) 56, 58 of extensible layer 42.

Indicia 46 may have any size or shape, and may be regular or irregular in shape. Suitable indicia 46 include, but are not limited to, any type of designs, marks, figures, identification codes, words, patterns, and the like. Example of indicia 46 include, but are not limited to, smiley faces, squares, waves, triangles, tetragons, pentagons, hexagons, circles, ellipses, crescent-shapes, teardrops, ob-round shapes or a mixture thereof.

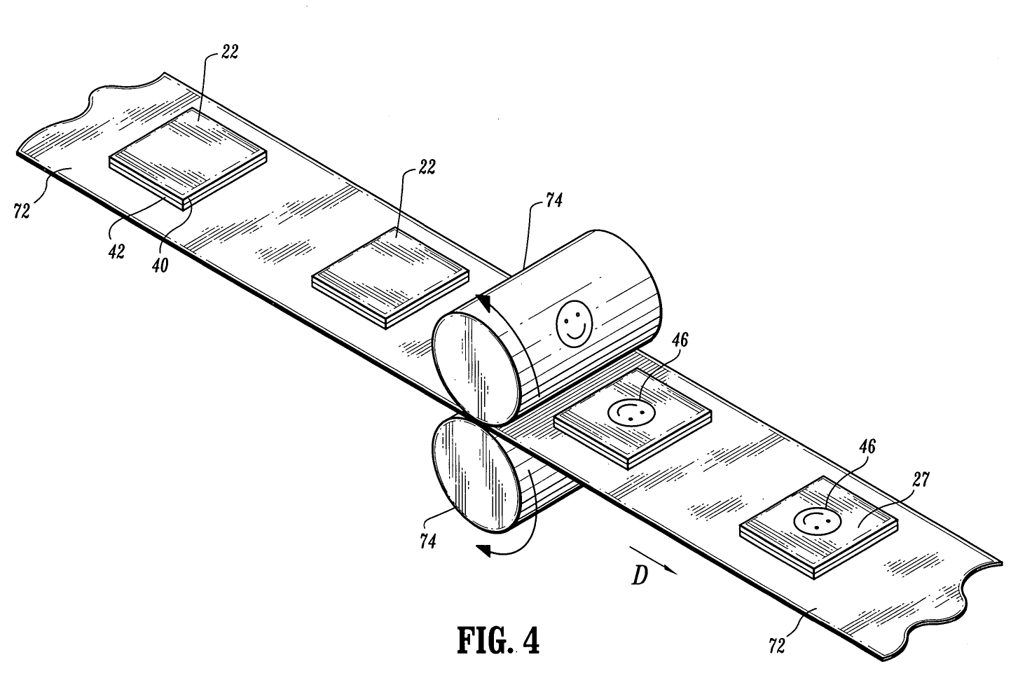

A method of manufacturing fasteners 22, similar to that described, in accordance with the principles of the present disclosure is provided. The process can best be understood by reference to an illustrative embodiment shown in FIG. 4. Initially, fasteners 22 include an elastic laminate of nonwoven layer 40 and extensible layer 44, which are provided. Fasteners 22 are conveyed on conveyer 72 in a direction D toward applicator 74. Elastic laminates that are suitable for use with the present invention are known in the art, such as, for example, FABRIFLEX available from Tredegar Film Products of Richmond, Va.

As fasteners 22 are conveyed, applicator 74 prints indicia such as smiley face 46 onto nonwoven layer 40 of fastener 22 along stretchable zone 27 thereof. Other means of applying indicia 46 to fasteners 22 are contemplated. For example, in addition to the roller application, it is possible to use any printing process known in the art, such as, for example, letterpress, lithography, gravure, silk screen, and the like. The inks used should be safe for human use and should not have environmentally deleterious effects. The inks chosen should, of course, be suitable for the intended printing process. Applicator 74 may comprise, for example, a roller, stamp, plate, ink jet or other means suitable for the purpose. Alternatively, fastener material having dynamic indicia already printed thereon can be obtained from a supplier.

After application of indicia 46 to fasteners 22, fasteners 22 travel on conveyer 72 in a direction D to a location for application to an absorbent article (not shown). Advantageously, indicia 46 are designed to change configuration so to indicate desired or non-desired fit of the absorbent article to a wearer when fastener 22 is stretched. It is further envisioned that indicia 46 may be applied to nonwoven layer 40 or extensible layer 42 prior to lamination (not shown) thereof.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. An absorbent article comprising:
at least one fastening tab, wherein a first crosswise portion of the fastening tab is extensible and includes a plurality of layers; and
indicia disposed with the first crosswise portion and being adapted to change configuration upon extension of the fastening tab,
wherein during extension of the fastening tab, the indicia changes from a first configuration to a second configuration.

2. An absorbent article according to claim 1, wherein the indicia changes from a first configuration that indicates a desired fit to a second configuration that indicates a non desired fit.

3. An absorbent article according to claim 1, further including a second crosswise portion of the fastening tab that is non-extensible.

4. An absorbent article according to claim 3, wherein the second crosswise portion does not contain the indicia.

5. An absorbent article according to claim 1, wherein the fastening tab includes an extensible layer adjacent at least one non-woven layer.

6. An absorbent article according to claim 5, wherein the indicia is placed intermediate the non-woven layers.

7. An absorbent article according to claim 5, wherein the indicia is placed adjacent one of the non-woven layers.

8. An absorbent article according to claim 5, wherein the indicia is printed upon at least one of the non-woven layers.

9. An absorbent article according to claim 5, wherein the indicia is printed upon the extensible layer.

10. An absorbent article according to claim 5, wherein the indicia is printed upon one of the at least two non-woven layers.

11. An absorbent article according to claim 1, wherein the fastening tab includes an extensible layer intermediate two non-woven layers.

12. An absorbent article according to claim 1, wherein the absorbent article is a diaper.

13. A fastener adapted for use with an absorbent article, the fastener comprising:
a first crosswise portion that is extensible and includes a non-woven layer and an extensible layer; and
indicia disposed with the non-woven layer and the extensible layer, and being adapted to change configuration upon extension of the fastener.

14. A fastener according to claim 13, wherein the indicia changes from a first configuration that indicates a desired fit to a second configuration that indicates a non desired fit.

15. A fastener according to claim 13, wherein the indicia is altered upon extension of the fastener.

16. An absorbent article comprising:
a pair of fasteners, wherein at least one of the fasteners includes indicia adapted to change configuration upon extension of the fastener so to indicate fit of the diaper to an individual,
wherein during extension of the fastening tab, the indicia changes from a first design that indicates a desired fit to a second design that indicates a non-desired fit.

17. An absorbent article according to claim 16, wherein the fastener includes an extensible layer adjacent at least one non-woven layer.

18. A fit-indicating diaper comprising:
a pair of fasteners, wherein at least one of the fasteners includes dynamic indicia;
wherein the dynamic indicia indicates a desired fit and a non-desired fit of the diaper to an individual.

19. A fit-indicating diaper according to claim 18, wherein each of the fasteners include at least one non-woven substrate layer and at least one extensible substrate layer.

20. A fit-indicating diaper according to claim 18, wherein the dynamic indicia is printed upon the at least one non-woven substrate layer.

21. A diaper according to claim 18, wherein the dynamic indicia is printed upon the at least one extensible substrate layer.

22. A method of manufacturing a fastening tab, the method comprising the steps of:
providing a fastening tab having at least one non-woven substrate layer and at least one extensible substrate layer; and
printing indicia on at least one of the non-woven substrate layer and the extensible substrate layer,
wherein the indicia is aligned along a crosswise stretchable zone of the fastening tab, and is adapted to change configuration upon extension of the fastening tab so to indicate fit of an absorbent article to an individual, and
wherein during extension of the fastening tab, the indicia changes from a first configuration that indicates a desired fit to a second configuration that indicates a non-desired fit.

23. A method according to claim 22, wherein the indicia changes color to indicate the desired and non-desired fit of the absorbent article to the individual.

24. A method according to claim 22, wherein the indicia changes shape to indicate the desired and non-desired fit of the absorbent article to the individual.
